(19) United States
(12) Patent Application Publication
(10) Pub. No.: US 2011/0190804 A1
(43) Pub. Date: Aug. 4, 2011

(54) FLUID INJECTION DEVICE

(75) Inventors: Hirokazu SEKINO, Chino-shi (JP); Takeshi SETO, Chofu-shi (JP); Hideki KOJIMA, Matsumoto-shi (JP)

(73) Assignee: SEIKO EPSON CORPORATION, Tokyo (JP)

(21) Appl. No.: 13/015,081

(22) Filed: Jan. 27, 2011

(30) Foreign Application Priority Data
Feb. 2, 2010 (JP) 2010-020956

(51) Int. Cl. A61B 17/3203

(52) U.S. Cl. 606/190

(57) ABSTRACT

A fluid injection device that injects a fluid to a biological tissue from an injection opening to excise the biological tissue, includes: a fluid injection means that injects the fluid from the injection opening; and a suction means that sucks at least one of the injected fluid and the excised biological tissue by periodically varying a negative suction pressure.

This application claims priority to Japanese Patent Application No. 2010-020956, filed on Feb. 2, 2010, the entirety of which is hereby incorporated by reference.

### BACKGROUND

1. Technical Field

The present invention relates to a technique for injecting a fluid from an injection opening.

2. Related Art

Recently, a surgical technique in which a fluid such as water or physiological saline is pressurized to inject the fluid to a surgical site during surgery, thereby excising a biological tissue by the pressure of the fluid has been developed. A fluid injection device employed for such a surgery is configured to inject a fluid from an injection opening provided at the tip of a nozzle, and an operator can excise a biological tissue at a surgical site by injecting the fluid to the surgical site by pointing the injection opening of the nozzle at the surgical site.

When the fluid injected from the injection opening accumulates around the surgical site, for example, the visibility of the surgical site is reduced and it is difficult to excise a biological tissue at a desired site, or the force of the injected fluid stream is decreased by the fluid accumulating at the surgical site, blood accumulating at the surgical site due to the excision, or the like, and the excision force is decreased. Moreover, there may arise a problem that the excised biological tissue (excised tissue) is scattered by the injected fluid, and for example, an excised malignant tumor reaches a biological tissue at a site other than the surgical site and causes an adverse effect, and so on. Accordingly, a technique for avoiding the occurrence of such a problem by providing a suction opening connected to a suction unit in the vicinity of an injection opening so as to suck a fluid, an excised tissue, or blood accumulating at a surgical site into the suction opening when bringing the injection opening close to the surgical site has been proposed (JP-A-6-90957).

However, when the proposed technique is used, there is a possibility that a non-excised biological tissue may be damaged depending on the condition of the surgical site by sucking the non-excised biological tissue at the surgical site along with the fluid or blood accumulating around the surgical site or the excised tissue. If the suction amount is reduced in order to avoid damaging the biological tissue in light of this possibility, the fluid injected from the injection opening or the excised tissue cannot be sufficiently sucked, and the above-mentioned problem may be caused.

### SUMMARY

An advantage of some aspects of the invention is to provide a technique for reducing damage caused by sucking a non-excised biological tissue while securing a necessary suction amount at the time of sucking a fluid injected from an injection opening, blood accumulating at a surgical site, and an excised biological tissue.

A fluid injection device according to an aspect of the invention has the following configuration. That is, the fluid injection device that injects a fluid to a biological tissue from an injection opening to excise the biological tissue includes: a fluid injection unit that injects the fluid from the injection opening; and a suction unit that sucks at least one of the injected fluid and the excised biological tissue by periodically varying a negative suction pressure.

The fluid injection device according to the aspect of the invention excises a biological tissue at a surgical site by the pressure of the fluid injected from the injection opening. The fluid accumulating at the surgical site by being injected in this manner and the excised biological tissue are sucked by the suction unit. The suction unit can periodically vary the negative suction pressure when the unit sucks the fluid at the surgical site.

The phrase “a negative suction pressure is periodically varied” as used herein means that a negative suction pressure is varied by an embodiment in which a high negative pressure and a low negative pressure are periodically repeated. The embodiment in which “a negative suction pressure is periodically varied” is not limited to the embodiment in which a high negative pressure value and a low negative pressure value are merely alternately repeated, and an embodiment in which a negative pressure value is continuously varied between a high negative pressure value and a low negative pressure value is also included.

In the case where the fluid is sucked by a predetermined negative suction pressure, there is no alternative but to set the negative suction pressure according to the amount of the fluid to be sucked, and a condition for sucking the fluid (the negative suction pressure in this case) cannot be freely changed. On the other hand, if the negative suction pressure can be periodically varied, a suction condition such as a value of the negative suction pressure or a varying cycle of the negative suction pressure can be changed. Therefore, even when a biological tissue is almost sucked along with the fluid under a certain suction condition, by changing the suction condition, the suction can be performed under an appropriate condition without fear of sucking the biological tissue while securing a necessary suction amount. In particular, the condition of the surgical site to which the fluid is injected is not uniform, and an optimal suction condition is different in various ways. Accordingly, by performing suction while periodically varying the negative suction pressure, the degree of freedom of selection of the suction condition can be increased to a large extent, and as a result, an optimal suction condition suitable for the surgical site can be selected.

The fluid injection device according to the above-mentioned aspect of the invention may be configured to eject the fluid in the form of a pulse flow from the injection opening, and when the suction condition for sucking the fluid is set, information relating to the amount of the fluid injected from the injection opening is obtained and the suction condition may be set based on the obtained information.

The phrase “the fluid is injected in the form of a pulse flow” as used herein means that the fluid is injected in a state where the flow amount of the fluid to be injected is accompanied by variation in the flow rate of the fluid. Accordingly, an embodiment in which the fluid is injected in the form of a pulse flow includes intermittent injection in which injection is performed while alternately repeating start and stop of the injection of the fluid, and it is only necessary that the flow amount of the fluid be such that the flow rate varies.

The suction amount of the fluid to be sucked varies in accordance with the amount of the injected fluid. Therefore, if the suction condition is set based on the information relating to the amount of the injected fluid, even when the fluid is injected in a state where the flow amount of the fluid is accompanied by variation in the flow rate of the fluid by injecting the fluid in the form of a pulse flow, the fluid can be appropriately sucked according to the variation in the injection amount.

The fluid injection device according to the above-mentioned aspect of the invention may be configured to suck the fluid at the surgical site while periodically repeating a sucking state and a non-sucking state.

According to this configuration, in the case where the fluid at the surgical site is sucked, it is only necessary to control start and stop of the suction of the fluid to be periodically repeated, and therefore, the control or the mechanism for varying the negative suction pressure can be simplified as compared with the case where a negative pressure is varied by a plurality of negative suction pressures. When it is in a situation where a biological tissue is easily sucked, there is a case where damage to the biological tissue cannot be sufficiently avoided only by decreasing the negative suction pressure. For example, when it is in a situation where the surgical site is intricate in a complicated manner and a negative suction pressure is easily applied to a biological tissue, there may arise a case where the suction of the biological tissue cannot be avoided only by decreasing the suction force. In such a situation, by sucking the fluid while alternately repeating a sucking state and a non-sucking state, the suction of a biological tissue can be avoided.

The fluid injection device according to the above-mentioned aspect of the invention may be configured such that a maximum negative pressure of the negative suction pressure which periodically varies can be set.

The maximum negative pressure of the negative suction pressure can directly determine the force of sucking a biological tissue, and also an effect of changing the setting thereof can be intuitively perceived. Accordingly, by permitting the maximum negative pressure of the negative suction pressure to be set, an appropriate suction condition can be easily set.

The fluid injection device according to the above-mentioned aspect of the invention may be configured such that a suction cycle which is a varying cycle of the negative suction pressure can be set.

The suction cycle is also one of the suction conditions that affect the ease of suction of a biological tissue as well as the above-mentioned maximum negative pressure of the negative suction pressure. Accordingly, by permitting the suction cycle to be set, an appropriate suction condition can be easily set.

The fluid injection device according to the above-mentioned aspect of the invention may be configured such that a suction time which is a time for providing a negative suction pressure larger than a predetermined negative pressure within the suction cycle of the negative suction pressure can be set.

The suction time is also one of the suction conditions that affect the ease of suction of a biological tissue as well as the above-mentioned maximum negative pressure and suction cycle. Accordingly, by permitting the suction time to be set, an appropriate suction condition can be easily set.

### BRIEF DESCRIPTION OF THE DRAWINGS

The invention will be described with reference to the accompanying drawings, wherein like numbers reference like elements.

FIG. 1 is an explanatory view showing a schematic structure of a fluid injection device according to an embodiment of the invention.

FIGS. 2A and 2B are explanatory views showing a detailed structure of an injection mechanism provided in a fluid injection device according to an embodiment of the invention.

FIGS. 3A and 3B are explanatory views showing a structure for sucking a fluid of a fluid injection device according to an embodiment of the invention.

FIG. 4 is an explanatory view showing a suction mechanism driving process for driving a suction mechanism performed by a suction controller according to an embodiment of the invention.

FIG. 5 is an explanatory view showing a manner of applying a negative pressure to a suction opening as a result of performing a suction mechanism driving process.

FIG. 6 is an explanatory view showing a manner of changing a condition for sucking a fluid by determining two parameters for suction to be given values.

FIGS. 7A to 7C are explanatory views illustrating a manner of preventing damage to a biological tissue when a fluid at a surgical site is sucked using a fluid injection device according to an embodiment of the invention.

FIG. 8 is an explanatory view showing a condition for sucking a fluid in a first modification example in which a negative pressure is applied also in an intermittent time.

FIG. 9 is an explanatory view showing a schematic structure of a fluid injection device according to a second modification example.

### DESCRIPTION OF EXEMPLARY EMBODIMENTS

Hereinafter, embodiments will be described in the following order for clarifying the contents of the invention.

A. Structure of Device

B. Suction Mechanism Driving Process according to Embodiment of Invention

C. Modification Examples

C-1. First Modification Example

C-2. Second Modification Example

C-3. Third Modification Example

### A. STRUCTURE OF DEVICE

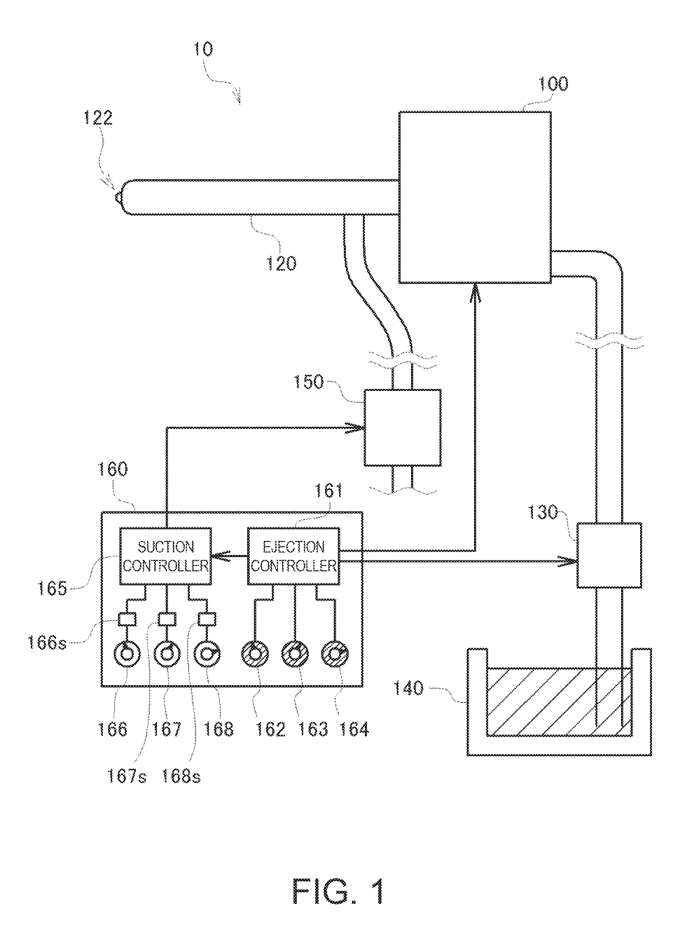

FIG. 1 is an explanatory view showing a schematic structure of a fluid injection device according to an embodiment of the invention. As shown in the drawing, a fluid injection device 10 according to this embodiment is provided with a fluid container 140 in which a fluid such as physiological saline or a drug solution is stored, a fluid supply means 130 that sucks the fluid from the fluid container 140, an injection mechanism 100 that pressurizes the fluid sucked by the fluid supply means 130 and delivers the fluid to a fluid injection pipe 120, a suction mechanism 150 for sucking the fluid injected by the fluid injection device 10 and the like, a controller 160 that controls the operation of the fluid injection device 10, and the like.

The fluid pressurized by the injection mechanism 100 passes through the fluid injection pipe 120 and is injected to a surgical site in the body from an injection opening 122 provided at the tip of the fluid injection pipe 120 so that a biological tissue at the surgical site can be excised by the pressure of the fluid. If the injected fluid or the like is left as such, a trouble for excision or visibility of the surgical site occurs, and therefore, suction openings (not shown) are provided in the vicinity of the injection opening 122. The suction openings are connected to the above-mentioned suction mechanism 150, and by applying a negative pressure to the suction openings by driving the suction mechanism 150, the fluid injected from the injection opening 122 to the surgical site, blood bleeding when a biological tissue is excised, the excised biological tissue (excised tissue), or the like can be sucked into the suction opening.

An operation of injecting the fluid or an operation of sucking the fluid performed by the fluid injection device 10 is controlled by the controller 160. As shown in FIG. 1, the controller 160 according to this embodiment is provided with an injection controller 161 that controls the operation of injecting the fluid performed by the fluid injection device 10 and a suction controller 165 that controls the operation of sucking the fluid performed by the fluid injection device 10. The injection controller 161 is connected to the fluid supply means 130 and the injection mechanism 100, and the suction controller 165 is connected to the suction mechanism 150.

The injection controller 161 changes the amount of the fluid to be supplied to the injection mechanism 100 by controlling the fluid supply means 130, and also controls the injection mechanism 100 to change the condition for the fluid injection performed by the injection mechanism 100. The injection mechanism 100 injects the fluid intermittently (or strongly and weakly in alternate manners). Therefore, the injection controller 161 is provided with a fluid supply amount setting dial 162 for setting the amount of the fluid to be supplied to the injection mechanism 100 by the fluid supply means 130, an injection amount setting dial 163 for setting the amount of the fluid to be injected per injection by the injection mechanism 100, and an injection cycle setting dial 164 for setting the injection cycle.

The suction mechanism 150 sucks the fluid into the suction openings intermittently (or strongly and weakly in alternate manners), and the suction controller 165 controls the magnitude of the negative pressure at the time of sucking, the suction cycle, and the suction duration (suction time). Therefore, the suction controller 165 is provided with a negative suction pressure setting dial 166 for setting the negative suction pressure, a suction cycle setting dial 167 for setting the suction cycle, and a suction time setting dial 168 for setting the suction time.

Incidentally, although the suction controller 165 is provided with three setting dials (a negative suction pressure setting dial 166, a suction cycle setting dial 167, and a suction time setting dial 168), an operator of the fluid injection device 10 may not always operate these three setting dials. The details will be described later, but what can be operated by the operator may be only two setting dials arbitrarily selected from the three setting dials. When the operator operates two setting dials, the setting of the remaining setting dial is determined by itself. Therefore, a selection switch is provided for each of the setting dials. That is, the negative suction pressure setting dial 166 is provided with a negative suction pressure selection switch 166s; the suction cycle setting dial 167 is provided with a suction cycle selection switch 167s; and the suction time setting dial 168 is provided with a suction time selection switch 168s. When the fluid suction condition for the suction mechanism 150 is changed, the operator selects two selection switches to be set among the selection switches 166s, 167s, and 168s, and operates the setting dials corresponding to the selected switches with the switches turned on. By doing this, the suction condition can reflect the set values of the operated setting dials.

The fluid injection device 10 according to this embodiment as described above adopts the injection mechanism 100 as follows so as to obtain a large excision force by injecting the fluid at a high pressure.

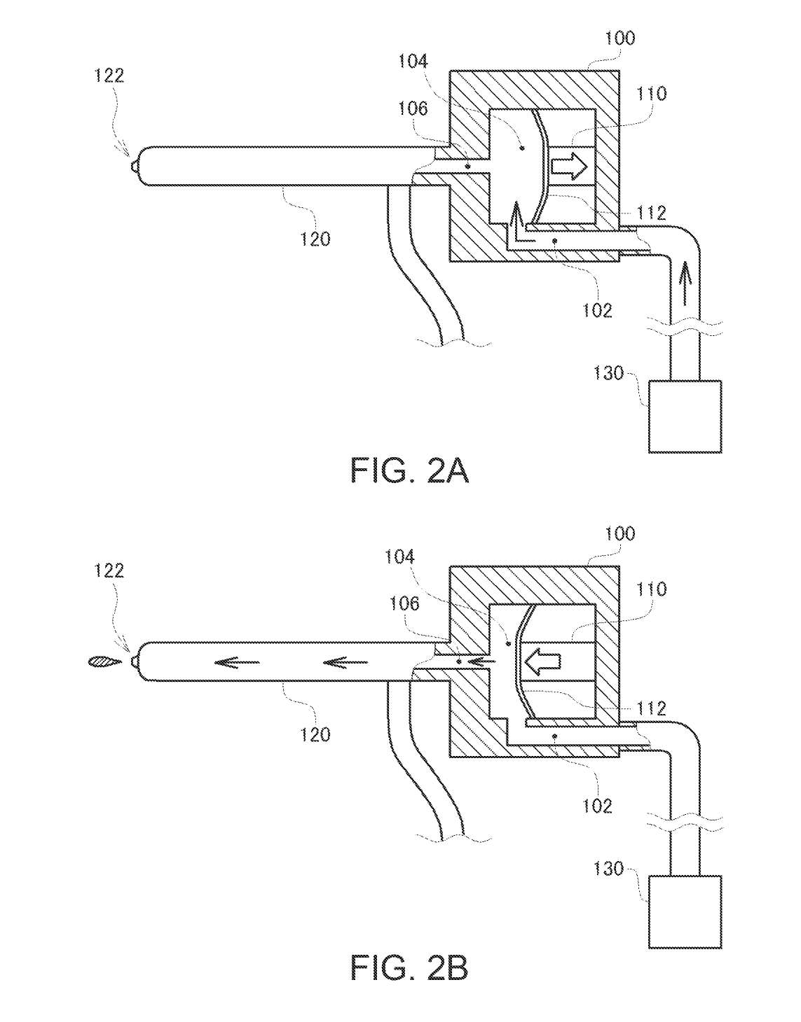

FIGS. 2A and 2B are explanatory views showing a structure of the injection mechanism 100 provided in the fluid injection device 10 according to this embodiment. As shown in FIG. 2A, the injection mechanism 100 is provided with a first connection tube 102 through which the fluid is supplied by the fluid supply means 130, a fluid chamber 104 filled with the fluid supplied from the fluid supply means 130, an injection pipe 106 through which the fluid is injected from the pressurizing chamber 104 to the fluid injection pipe 120, and the like. To the pressurizing chamber 104, a piezoelectric element 110 is connected via a diaphragm 112, and a voltage is applied to the piezoelectric element 110 to stretch or contract the piezoelectric element, whereby the diaphragm 112 is driven and the volume in the inside of the pressurizing chamber 104 can be changed.

When the fluid is injected, first, as shown in FIG. 2A, a voltage is applied to the piezoelectric element 110 to contract the piezoelectric element 110, whereby the volume of the pressurizing chamber 104 is expanded. At this time, the fluid is supplied to the pressurizing chamber 104 by the fluid supply means 130, and therefore, when the volume of the pressurizing chamber 104 is expanded, the pressurizing chamber 104 is filled with the fluid. Subsequently, as shown in FIG. 2B, the piezoelectric element 110 is stretched to compress the pressurizing chamber 104. At this time, to the fluid chamber 104, two channels, i.e. the injection pipe 106 and the first connection tube 102 are connected. These channels are formed to have a small diameter, and therefore, by compressing the fluid chamber 104 with the piezoelectric element 110, the pressure of the fluid in the fluid chamber 104 can be sufficiently increased. By employing this pressure, the fluid in the fluid chamber 104 is strongly pushed out in the direction of the injection pipe 106, and as a result, the fluid can be injected vigorously from the injection opening 122 connected to the injection pipe 106. Incidentally, the fluid in the fluid chamber 104 is pushed out not only into the injection pipe 106, but also into the first connection tube 102. However, the ease of flowing of the fluid into the respective channels (so-called inertance) is determined by the length of the channel, the cross-sectional area of the channel, and the like, and therefore, if the lengths and the cross-sectional areas of the first connection tube 102 and the injection pipe 106 are appropriately set, the amount of the fluid flowing into the first connection tube 102 can be suppressed less than the amount of the fluid flowing into the injection pipe 106. By doing this, the greater part of the fluid in the fluid chamber 104 can be pushed out into the injection pipe 106, and the fluid can be injected at a high speed from the injection opening 122 connected to the injection pipe 106.

After the fluid is injected in this manner, if the piezoelectric element is contracted again (see FIG. 2A) and thereafter the piezoelectric element 110 is stretched (see FIG. 2B), the fluid can be injected again. By repeating such an operation, the fluid droplets can be vigorously injected repeatedly in the fluid injection device 10 according to this embodiment, and by utilizing a high pressure generated when the vigorously injected fluid droplets collide against a target, an ability to excise a biological tissue is increased.

When a biological tissue at a surgical site is excised by injecting the fluid in this manner, there is a possibility that the fluid, blood, the excised tissue, and the like are accumulating at the surgical site to reduce the visibility of the surgical site, or the fluid and the like accumulating at the surgical site become an obstacle to decrease an ability to excise a biological tissue. Accordingly, the fluid injection device 10 according to this embodiment is configured as follows so as to suck the fluid and the like accumulating at the surgical site.

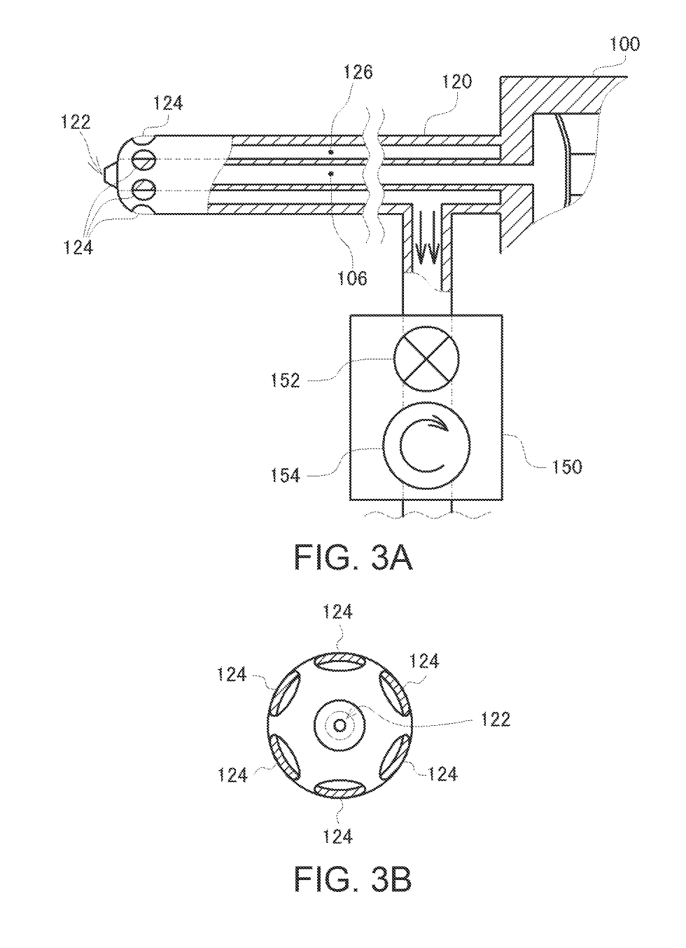

FIGS. 3A and 3B are explanatory views showing a structure for sucking the fluid and the like of the fluid injection device 10. As shown in FIG. 3A, a plurality of suction openings 124 are arranged in the vicinity of the injection opening 122 provided at the tip of the fluid injection pipe 120, and the plurality of these suction openings 124 are disposed surrounding the injection opening 122 taking the injection opening 122 as the center (see FIG. 3B).

The plurality of the suction openings 124 are disposed apart from the injection opening 122 toward the injection mechanism 100 on the side surface of the fluid injection pipe 120. Further, the fluid injection pipe 120 according to this embodiment is formed to have a double tube structure, and the inside of the inner tube serves as the injection pipe 106 connecting the injection mechanism 100 to the injection opening 122, and a region sandwiched between the inner tube and the outer tube serves as a suction flow passage 126 connecting the suction openings 124 to the suction mechanism 150.

The suction flow passage 126 is connected to a suction means 154 via a shut-off valve 152 in the suction mechanism 150, and by opening or closing the shut-off valve 152 while operating the suction means 154, the operator can switch whether or not a negative pressure generated by the suction means 154 is applied to the suction openings 124.

In the fluid injection device 10 according to this embodiment having such a configuration, when the fluid and the like accumulating at the surgical site are sucked, the suction means 154 in the suction mechanism 150 is operated in a state where the injection opening 122 is brought close to the surgical site. Then, a negative pressure is applied to the suction openings 124 disposed around the injection opening 122 (see FIG. 3B), whereby the fluid and the like accumulating around the injection opening 122 (i.e., the surgical site) are sucked.

Here, in order to prevent the fluid and the like from accumulating around the surgical site, it is necessary to suck the injected fluid, blood bleeding when a biological tissue is excised, and also the excised biological tissue, and it is demanded that a sufficient suction amount be secured at the time of sucking the fluid and the like. However, if the force of sucking the fluid and the like is too high, there is a fear of sucking a non-excised biological tissue along with the fluid and the like accumulating around the surgical site to damage the biological tissue. In order to avoid such a situation, it is conceivable that the biological tissue is made difficult to be sucked by reducing the force of sucking the fluid and the like. However, by doing this, the fluid injected to the surgical site and the like cannot be sufficiently sucked, and therefore, a problem that the visibility of the surgical site is reduced due to the accumulation of the fluid and the like at the surgical site or the excision ability of the fluid injection device 10 is decreased may be caused. Accordingly, in order to solve the above-mentioned two problems, the fluid injection device 10 according to this embodiment is configured to suck the fluid and the like accumulating at the surgical site by a method as follows.

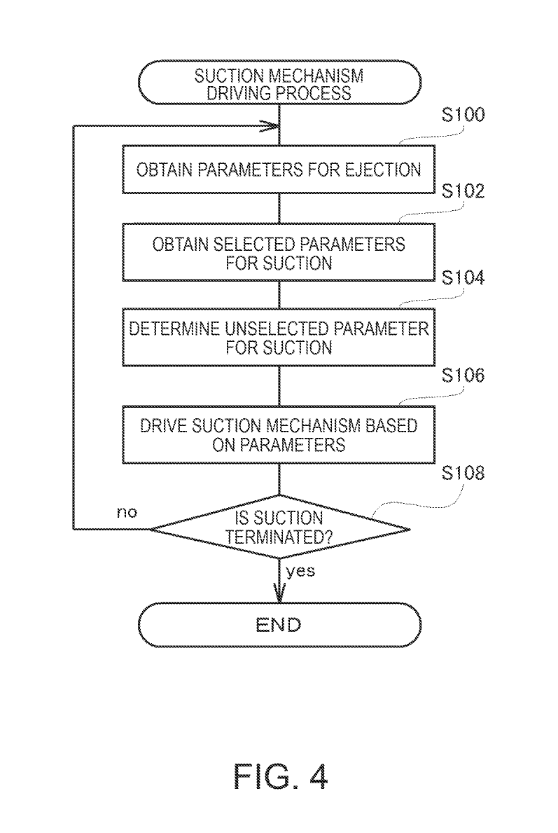

FIG. 4 is a flowchart showing a suction mechanism driving process according to this embodiment. As described above, the suction controller 165 according to this embodiment is connected to the injection controller 161 (see FIG. 1), and the suction controller 165 starts a suction mechanism driving process described below after confirming via the injection controller 161 that the fluid is started to be injected.

When the suction mechanism driving process is started, first, parameters for the injection are obtained from the injection controller 161 (Step S100). Here, the parameters for the injection mean values set by the fluid supply amount setting dial 162, the injection amount setting dial 163, and the injection cycle setting dial 164 of the injection controller 161 as described above with reference to FIG. 1. Since the injection controller 161 and the suction controller 165 are connected to each other, the suction controller 165 can obtain the parameters for the injection via the injection controller 161.

Subsequently, parameters for the suction are obtained (Step S102). Here, the parameters for the suction mean values set by the negative suction pressure setting dial 166, the suction cycle setting dial 167, and the suction time setting dial 168 of the suction controller 165 as described above with reference to FIG. 1. As described above, only two setting dials selected by an operator from these three setting dials 166, 167, and 168 may be permitted to be set, and when two parameters are set, the remaining parameter is determined by itself. Accordingly, when the parameters for the suction are obtained, parameters “selected” by the selection switches 166s, 167s, and 168s are obtained (Step S102).

After the “selected” parameters for the suction are obtained in this manner (Step S102), based on the parameters for the injection obtained in the Step S100 and the two parameters for the suction obtained in the Step S102, the parameter which is not selected by the operator is determined (Step S104). Here, the mechanism for determining the remaining parameter (unselected parameter) based on the two parameters selected by the operator is as follows.

First, the parameters for the injection obtained in the Step S100 of the suction mechanism driving process indicate the amount of the fluid supplied to the injection mechanism 100 by the fluid supply means 130, the amount of the fluid injected per injection by the injection mechanism 100, and the injection cycle of the injection mechanism 100, and by obtaining these parameters for the injection, the amount of the fluid to be injected by the fluid injection device 10 in a given time can be determined. In order to prevent the fluid from accumulating at the surgical site, it is only necessary to suck the injected fluid, and also to suck blood bleeding when a biological tissue is excised by injecting the fluid, the excised biological tissue, and the like. Therefore, if the injection amount of the fluid in a given time is determined, also the amount of the fluid and the like to be sucked in a given time can be determined in association with the injection amount of the fluid. The amount of the fluid and the like to be sucked in a given time can be deduced from the injection amount of the fluid in a given time. The amount of the excised tissue and the amount of blood bleeding due to the excision vary depending also to the condition of the surgical site, and therefore, it is preferred that the operator can adjust the amount of the fluid and the like to be sucked in a given time.

On the other hand, if the three parameters for the suction (negative suction pressure, suction cycle, and suction time) are determined, the suction amount is determined. Accordingly, under a condition that the injection amount of the fluid is determined by determining the parameters for the injection, also the suction amount of the fluids and the like to be sucked in a given time is determined, and therefore, if the operator sets two parameters, a set value of the remaining one parameter is determined to be a certain value based on the necessary suction amount. Hereinafter, description will be made by providing an additional explanation for this point.

It is assumed that first, the suction cycle is selected from the three parameters for the suction (negative suction pressure, suction cycle, and suction time) and set to a desired value. Since the suction amount in a given time is determined based on the injection amount, the amount of the fluid and the like to be sucked in the set cycle is determined. Subsequently, if the suction time in the cycle is set to a certain time, the negative suction pressure is determined based on the amount of the fluid and the like to be sucked in the cycle. For example, in the case where the suction time in the cycle is set to a relatively long time, the negative suction pressure can be set to a relatively low value. On the other hand, in the case where the suction time in the cycle is set to a relatively short time, the negative suction pressure can be set to a relatively high value.

In the Step S104 of the suction mechanism driving process shown in FIG. 4, the remaining “unselected” parameter is determined based on the two parameters selected by the operator as described above. After all of the three parameters for the suction (negative suction pressure, suction cycle, and suction time) are determined in this manner, the suction mechanism 150 is driven based on these parameters for the suction (Step S106).

Subsequently, the fluid injection device 10 determines whether or not the fluid suction is terminated (Step S108). The fluid injection device 10 according to this embodiment is configured to terminate the fluid suction simultaneously with the termination of the fluid injection. Accordingly, in the Step S108, whether or not the fluid suction is terminated is determined by confirming whether or not the fluid injection is terminated via the injection controller 161 that controls the fluid injection (Step S108).

In the case where the fluid injection is still continued, it is determined that the suction of the fluid and the like is also continued (Step S108: no), and the step returns to Step S100 of the suction mechanism driving process again and the above-mentioned series of steps are performed. In the course of repeating these steps, when the operator terminates the fluid injection, it is determined that the suction is terminated (Step S108: yes), and the suction mechanism driving process is terminated. Incidentally, there may be a case where even if the fluid injection is terminated, suction is desired to be continued because blood or an excised tissue accumulates. Accordingly, the fluid injection device may be configured such that suction is terminated after a lapse of a given time from the termination of the fluid injection, and the operator can input a command to terminate the suction.

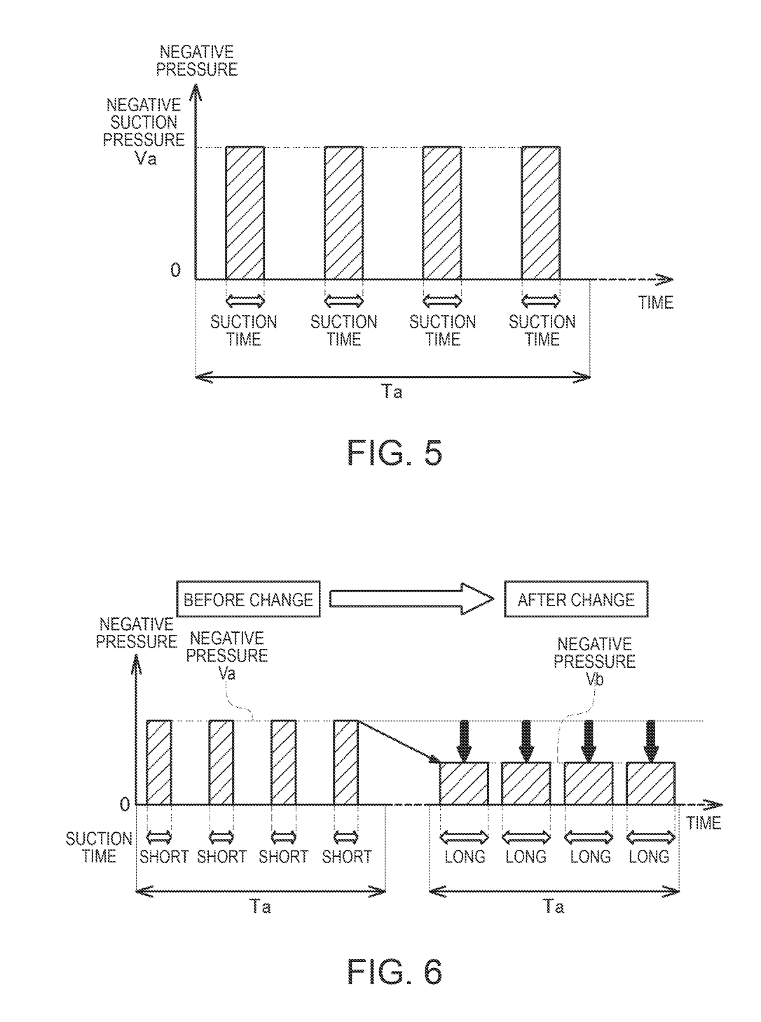

FIG. 5 is an explanatory view showing a manner of applying a negative pressure to the suction openings as a result of performing the suction mechanism driving process according to this embodiment. Incidentally, in FIG. 5, a manner in which a negative pressure is applied to the suction openings 124 in the case where the suction cycle and the negative suction pressure among the three parameters for the suction are set by the operator.

It is assumed that the suction cycle is set to 4 times per unit time Ta and the negative suction pressure is set to Va by the operator. At this time, since the fluid amount to be sucked by the above-mentioned suction mechanism driving process has been determined, the suction time which is the remaining parameter for the suction is determined by itself based on the suction amount of the fluid, the suction cycle, and the negative suction pressure. As a result, as shown in FIG. 5, a negative pressure is to be applied to the suction openings 124 only for a predetermined time per set cycle (4 times/Ta) based on the negative suction pressure (Va) set by the operator. Incidentally, the area of the shaded region in FIG. 5 corresponds to the suction amount of the fluid and the like. Here, as described above, the fluid injection device 10 according to this embodiment is configured to be able to set two parameters among the three parameters for the suction (negative suction pressure, suction cycle, and suction time) to given values, and by determining the values of the two parameters, the condition for sucking the fluid can be changed as described below.

FIG. 6 is an explanatory view showing a manner of changing the condition for sucking the fluid and the like by determining two parameters for the suction to be given values. Incidentally, in FIG. 6, two suction conditions in the case where the suction cycle and the negative suction pressure are determined to be given values are shown. In the two suction conditions shown in FIG. 6, the amount of the fluid and the like to be sucked is the same.

On the left side of FIG. 6, a suction condition in the case where the negative suction pressure (Va) and the suction cycle (4 times/Ta) are determined to be the same as the case shown in FIG. 5 is shown (in the drawing, indicated by “before change”). Under this condition, since the negative suction pressure Va is a relatively high value, the suction time becomes short in order to meet the predetermined suction amount. As a result, the suction condition is such that a cycle in which the suction is performed at a high negative pressure for a short time is repeated. On the other hand, on the right side of FIG. 6, a suction condition in the case where the suction cycle is determined to be the same as the above-mentioned case of “before change” (4 times/Ta) and the negative suction pressure is determined to be a negative suction pressure (Vb) lower than that in the case of “before change” (in the drawing, indicated by “after change”) is shown. Under this condition, since the negative suction pressure Vb is lower than the negative pressure Va, the suction time becomes long in order to meet the predetermined suction amount. As a result, the suction condition can be changed such that a cycle in which the suction is performed at a lower negative pressure for a longer time as compared with the suction condition indicated by “before change” is repeated.

As described above, in the fluid injection device 10 according to this embodiment, by determining the suction cycle and the negative suction pressure to given values, the suction condition for sucking the fluid and the like can be changed. The suction condition is not limited to the example shown in FIG. 6, and the values to be set of the suction cycle and the negative suction pressure can be changed to values desired by the operator. Therefore, according to the magnitude of the set values, the suction condition for sucking the fluid and the like can be changed subtly. Further, the parameters to be selected are not limited to the suction cycle and the negative suction pressure, and the operator can select arbitrary two parameters from the three parameters for the suction (negative suction pressure, suction cycle, and suction time) and can freely set the values of the selected parameters. Accordingly, in the fluid injection device 10 according to this embodiment, the suction condition can be changed variously even if the suction amount of the fluid and the like is the same. This results in exhibiting an extremely large effect when surgery in which a biological tissue is excised is performed. Hereinafter, this point will be described.

First, when surgery in which a biological tissue is excised is performed, various conditions of the surgical site are conceived. For example, if focus is placed on the hardness of the biological tissue at the surgical site, there are a hard tissue and a soft tissue. If focus is placed on the size of the surgical site, there are a large surgical site where tissues at the surgical site are not so intricate, and also a narrow surgical site where tissues are intricate in a complicated manner. Since the condition of the surgical site is not the same as described above, it is considered that the suction condition capable of sucking the fluid and the like accumulating the surgical site without damaging the surgical site is also changed variously. In view of this, in the fluid injection device 10 according to this embodiment, since the suction condition for sucking the fluid and the like at the surgical site can be changed variously, an optimal suction condition according to the condition of the surgical site can be selected. As a result, the fluid and the like accumulating at the surgical site can be sucked while avoiding damaging a non-excised biological tissue at the surgical site as much as possible.

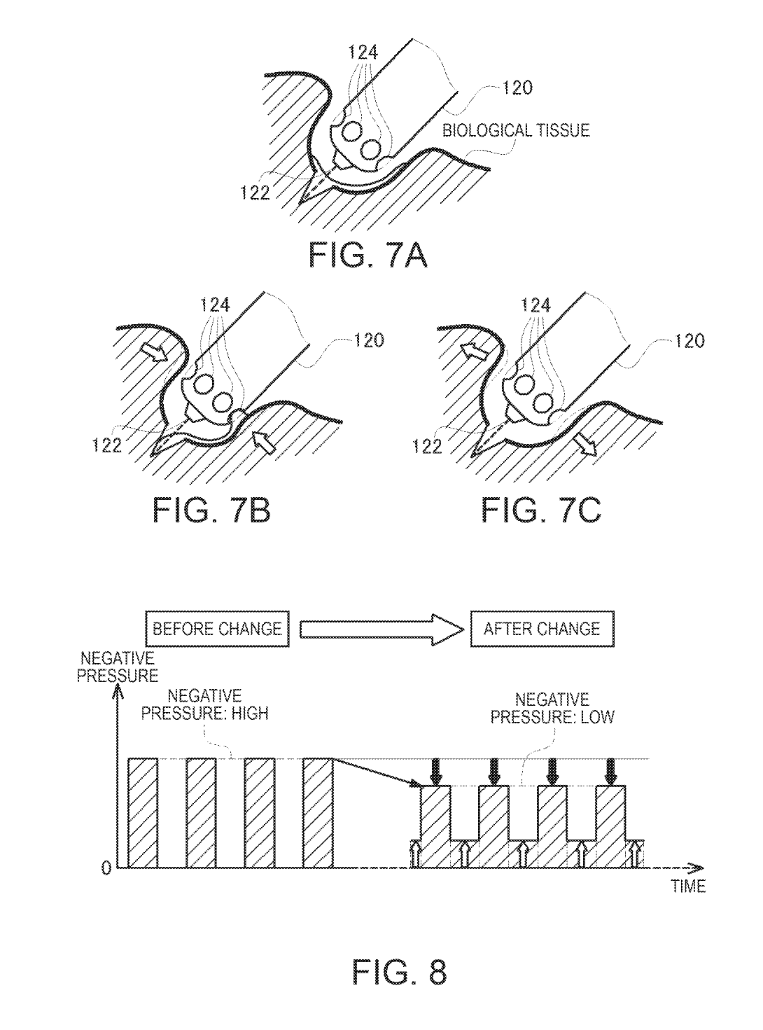

FIGS. 7A to 7C are explanatory views illustrating a manner of preventing damage to a non-excised biological tissue as much as possible when the fluid and the like at the surgical site are sucked using the fluid injection device according to this embodiment. Incidentally, the example shown in FIGS. 7A to 7C corresponds to the case where the surgical site is relatively narrow such as a region where biological tissues are intricate in a complicated manner.

When the fluid is injected from the injection opening 122 to excise a biological tissue at a surgical site, as shown in FIG. 7A, the fluid accumulates around the surgical site, and therefore, the device is operated such that the fluid and the like are sucked into the suction openings 124 simultaneously with the injection of the fluid. At this time, in a state where the surgical site is narrow and the biological tissue is present very close to the suction openings 124, as shown in FIG. 7B, not only the fluid and the like at the surgical site, but also the biological tissue close to the suction openings 124 are drawn to the suction openings 124 by sucking. In such a situation, for example, when the suction time is set to relatively short, even if the biological tissue at the surgical site is drawn to the suction openings 124 by sucking, the suction time is terminated soon, and therefore, as shown in FIG. 7C, the biological tissue moves away from the suction openings 124. Accordingly, damage to the biological tissue by being sucked into the suction openings 124 can be avoided.

As described above, in the fluid injection device 10 according to this embodiment, by permitting an optimal suction condition to be selected according to the condition of the surgical site, the fluid and the like accumulating at the surgical site can be sucked while avoiding damage to the non-excised biological tissue at the surgical site as much as possible. Even in the case where the suction condition is changed by changing the parameters for the suction, the suction amount of the fluid and the like is maintained, and therefore, a problem that the visibility of the surgical site is reduced due to the accumulation of the injected fluid and the like at the surgical site or the ability to excise a biological tissue is decreased due to the fluid and the like accumulating at the surgical site which becomes an obstacle to the excision is not caused.

### C. MODIFICATION EXAMPLES

In the above embodiment, several modification examples are included. Hereinafter, these modification examples will be briefly described. In the following modification examples, constituent members similar to those in the above embodiment are denoted by the same reference numerals, and the detailed description thereof will be omitted.

### C-1. First Modification Example

In the fluid injection device 10 according to the above embodiment, it is described that a negative pressure is applied to the suction openings 124 only in a suction time determined by the parameters for the suction. However, the fluid injection device 10 may be configured such that a negative pressure having a magnitude different from that of the negative pressure applied in the suction time in the above embodiment is applied to the suction openings 124 for a time when the negative pressure is not applied to the suction openings 124 between the suction times (hereinafter referred to as “intermittent time”) in the above embodiment.

FIG. 8 is an explanatory view showing a change in a suction condition in the case where a negative pressure is applied also in an intermittent time. Incidentally, on the left side of the FIG. 8, a suction condition in the case where a negative pressure is not applied in the intermittent time (in the drawing, indicated by “before change”) is shown, and on the right side of FIG. 8, a suction condition in the case where only a small negative pressure is applied also in the intermittent time (in the drawing, indicated by “after change”) is shown.

By applying a negative pressure to the suction openings 124 also in the intermittent time, the suction amount of the fluid is increased as compared with the case where a negative pressure is not applied in the intermittent time. Accordingly, if the same amount of the fluid is sucked, the amount of the fluid to be sucked in the suction time in the related art can be reduced by the increment of the amount of the fluid to be sucked in the intermittent time. As a result, as shown in FIG. 8, in the case where a negative pressure is applied also in the intermittent time (after change), the negative suction pressure applied in the suction time in the case of “before change” can be suppressed low as compared with the case where a negative pressure is not applied in the intermittent time (before change). By suppressing the negative suction pressure low in this manner, a load imposed on a non-excised biological tissue can be reduced when the fluid and the like at the surgical site are sucked, and therefore, damage to the biological tissue can be further suppressed.

### C-2. Second Modification Example

In the fluid injection device 10 according to the above embodiment and first modification example, it is described that the fluid injection pipe 120 has a double tube structure, the suction openings 124 are provided at the tip of the fluid injection pipe 120, a region sandwiched between the inner tube and the outer tube of the fluid injection pipe 120 is used as the suction flow passage 126, and therefore, the fluid injection pipe 120 has both of a fluid injection function and a fluid suction function. However, the fluid injection pipe 120 is exclusively used for injecting the fluid, and a suction pipe may be provided for sucking the fluid separately from the fluid injection pipe 120.

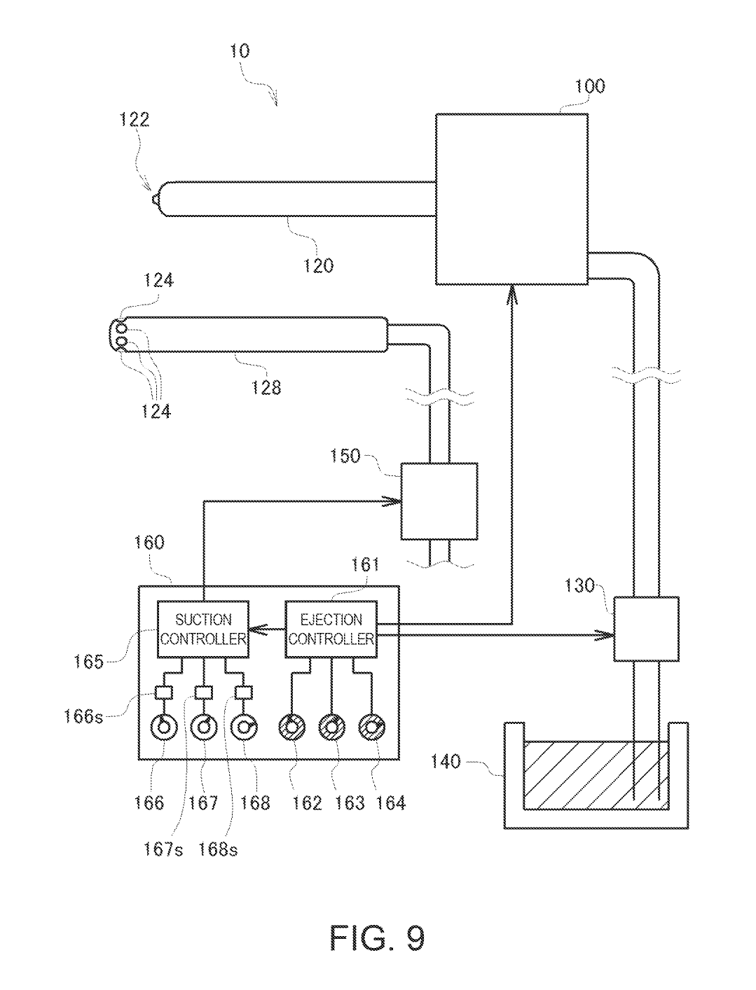

FIG. 9 is an explanatory view showing a schematic structure of the fluid injection device according to the second modification example. As shown in the drawing, in the fluid injection device 10 according to the second modification example, a suction pipe 128 for sucking the fluid is provided separately from the fluid injection pipe 120, and the suction openings 124 are provided on the side surface of the suction pipe 128 in the vicinity of the tip thereof. The suction openings 124 are connected to the suction mechanism 150 via the suction flow passage 126 (not shown) provided in the inside of the suction pipe 128. The position where the suction openings 124 are provided is not limited to the above position.

In the fluid injection device 10 according to the second modification example, the operator can freely move the suction pipe 128 while excising a biological tissue at a surgical site by pointing the tip of the fluid injection pipe 120 in a given direction. Accordingly, when the fluid and the like accumulating at the surgical site are sucked, it becomes possible to point the suction openings 124 at the surgical site at various angles. As a result, for example, at a surgical site where biological tissues are intricate in a complicated manner, by pointing the suction openings 124 at the surgical site at a more appropriate angle, the fluid and the like accumulating in such a region that the fluid therein is difficult to be sucked can be efficiently sucked. Even in the case where a non-excised biological tissue is drawn by sucking while excising a biological tissue at the surgical site, only the suction pipe 128 may be moved away from the surgical site. Therefore, there is no need to interrupt the ongoing surgery on the way, and accordingly, it is possible to avoid loss of time for surgery.

### C-3. Third Modification Example

In the above embodiment, first modification example, and second modification example, it is described that the operator can select two parameters from the three parameters for the suction (negative suction pressure, suction cycle, and suction time) and set them to given values. However, the parameters that can be set by the operator may be only the negative suction pressure and the suction cycle, and the suction time may be determined by itself so as to meet the suction amount.

Among the three parameters for the suction, the negative suction pressure and the suction cycle are parameters, the effect of which on the suction condition is more easily perceived intuitively than that of the suction time, and therefore, it is considered that if the negative suction pressure and the suction cycle are permitted to be set, the suction condition can be generally brought close to a suction condition desired by the operator. Therefore, at the time of setting the parameters for the suction, the negative suction pressure and the suction cycle are permitted to be set, and the suction time is allowed to be set automatically. By doing this, a necessary structure for setting the suction time (the suction time setting dial 168 and the suction time selection switch 168s) can be omitted, and also the control for determining the suction condition performed by the suction controller 165 can be simplified.

The fluid injection device according to this embodiment is described in the above, however, the invention is not limited to all of the above-mentioned embodiment and modification examples, and is applicable to various embodiments within a scope that does not deviate from the gist of the invention. In this embodiment, it is described that the fluid is injected in the form of a pulse by the injection mechanism, however, the form of the fluid to be injected is not limited thereto, and the fluid may be injected in any form.

What is claimed is:

1. A fluid injection device that injects a fluid to a biological tissue from an injection opening to excise the biological tissue, comprising:
a fluid injection unit that injects the fluid from the injection opening; and
a suction unit that sucks at least one of the injected fluid and an excised biological tissue by periodically varying a negative suction pressure.

2. The fluid injection device according to claim 1, wherein
the fluid injection unit injects the fluid in the form of a pulse flow from the injection opening, and further, the fluid injection unit includes:
an injection amount obtaining unit that obtains information relating to the amount of the fluid injected from the injection opening; and
a suction condition setting unit that sets a suction condition based on the obtained information relating to the amount of the fluid injected from the injection opening.

3. The fluid injection device according to claim 1, wherein
the suction unit is a unit that periodically repeats start and stop of suction.

4. The fluid injection device according to claim 1, further comprising a maximum negative pressure setting unit that sets a maximum negative pressure which is a maximum value of the negative suction pressure.

5. The fluid injection device according to claim 1, further comprising a suction cycle setting unit that sets a suction cycle which is a varying cycle of the negative suction pressure.

6. The fluid injection device according to claim 1, further comprising a suction time setting unit that sets the suction time which is a time for providing a negative suction pressure larger than a predetermined negative pressure within the suction cycle.
